(19) United States
(12) Patent Application Publication
(10) Pub. No.: US 2009/0005450 A1
(43) Pub. Date: Jan. 1, 2009

(54) Use of creatine compounds for the treatment of eye disorders

(76) Inventor: Belinda Tsao Nivaggioli, Atherton, CA (US)

Correspondence Address: LAHIVE & COCKFIELD, LLP;FLOOR 30, SUITE 3000, ONE POST OFFICE SQUARE, BOSTON, MA, 02109, US

(21) Appl. No.: 12/082,240

(22) Filed: Apr. 9, 2008

Related U.S. Application Data
(60) Provisional application No. 60/922,460, filed on Apr. 9, 2007.

(51) Int. Cl. A61K 31/195; A61P 27/02

(52) U.S. Cl. 514/565

(57) ABSTRACT

The present invention provides methods of treating an eye disorder (i.e., glaucoma, macular degeneration, diabetic retinopathy, macular edema, ocular rosacea, amblyopia, cataracts, dry eye, iritis, retinitis pigmentosa, uveitis etc.) by administering a creatine compound to a subject.

### RELATED APPLICATIONS

This application claims priority to U.S. Provisional Application Ser. No.: 60/922,460, filed on Apr. 9, 2007, the entire contents of which are hereby incorporated herein by reference.

### BACKGROUND OF THE INVENTION

The creatine kinase/creatine phosphate energy system is one component of an elaborate energy-generating system found in tissue with high and fluctuating energy requirements. The components of the creatine energy system include the enzyme creatine kinase, the substrates creatine and creatine phosphate, and the transporter of creatine. The reaction catalyzed by creatine kinase is: MgADP+PCr⁼+H⁺ MGATP⁼+Cr. Some of the functions associated with this system include efficient regeneration of energy in cells with fluctuating and high energy demands, energy transport to different parts of the cell, phosphoryl transfer activity, ion transport regulation, and involvement in signal transduction pathways. In addition, the creatine kinase system is very active in the retina, the heart, the muscles and the brain.

Creatine is a compound which is naturally occurring and is found in mammalian brain and other excitable tissues, such as skeletal muscle, retina and heart. Its phosphorylated form, creatine phosphate, also is found in the same organs and is the product of the creatine kinase reaction utilizing creatine as a substrate. Creatine phosphate is one of the highest energy generating compounds in the cell and creatine is an excellent stimulant of oxidative phosphorylation and high energy production. Creatine and creatine phosphate can be synthesized relatively easily and are believed to be non-toxic to mammals. Creatine, creatine phosphate and the enzymes that use them as substrates, i.e. the creatine kinases represent an efficient system for the rapid regeneration of energy. Kaddurah-Daouk et al. (WO 92/08456; WO 90/09192; U.S. Pat. No. 5,321,030; and U.S. Pat. No. 5,324,731) describe methods of inhibiting the growth, transformation and/or metastasis of mammalian cells using related compounds. Examples of compounds described by Kaddurah-Daouk et al. include cyclocreatine, β-guandidino propionic acid, homocyclocreatine, 1-carboxymethyl-2-iminohexahydropyrimidine, guanidino acetate and carbocreatine. These same inventors have also demonstrated the efficacy of such compounds for combating viral infections (U.S. Pat. No. 5,321,030). Elebaly in U.S. Pat. No. 5,091,404 discloses the use of cyclocreatine for restoring functionality in muscle tissue. Cohn in PCT publication No. WO 94/16687 described a method for inhibiting the growth of several tumors using creatine and related compounds. Kaddurah-Daouk et al.(WO 96/14063) reported on the neuroprotective effect of creatine compounds especially against neurodegenerative diseases such as Huntington's, Parkinson's, ALS, and Alzheimer's.

### SUMMARY OF THE INVENTION

In one embodiment, the present invention pertains, at least in part, to a method for treating an eye disorder in a subject by administering an effective amount of a creatine compound, such that the eye disorder is treated.

In another embodiment, the invention pertains to a method for modulating energy in the eye of a subject by administering an effective amount of a creatine compound. In a further embodiment, the invention pertains to a method of treating glaucoma in a subject by administering an effective amount of a creatine compound.

The present invention also pertains, at least in part, to a pharmaceutical composition comprising an effective amount of a creatine compound and a pharmaceutically acceptable carrier. In a further embodiment, the invention pertains in part to a pharmaceutical composition suitable for opthalmic administration.

Examples of the eye disorders suitable for treatment with creatine compounds of the present invention include, but are not limited to, glaucoma, macular degeneration, diabetic retinopathy, age-related macular degeneration, macular edema, ocular rosacea, amblyopia, cataracts, dry eye, iritis, retinitis pigmentosa and uveitis.

### DETAILED DESCRIPTION OF THE INVENTION

### I. Methods

The present invention pertains, at least in part, to a method for treating an eye disorder in a subject by administering an effective amount of a creatine compound. The eye disorder may be treated though the modulation of the energy in the eye, e.g., by the modulation of creatine kinase.

The present invention also pertains, at least in part, to a method for modulating energy in an eye of a subject by administering an effective amount of a creatine compound. The modulation of the energy in the eye may occur through the modulation of creatine kinase activity.

Additionally, the invention pertains, at least in part, to a method of treating glaucoma in a subject by administering an effective amount of a creatine compound. Glaucoma may be treated through modulation of the energy in the eye, e.g., by the modulation of creatine kinase.

The term “treated,” “treating” or “treatment” includes therapeutic and/or prophylactic treatment of eye disorders. The treatment includes the diminishment or alleviation of at least one symptom associated with an eye disorder. For example, treatment can be diminishment of one or several symptoms of the eye disorder or complete eradication of the eye disorder.

The term “eye disorder” includes disorders of the eye which can result in, for example, the reduction or loss of vision, degeneration of areas of the eye (e.g. macular area of the retina, cornea, conjuntiva, uvea, lens, etc.) or eye inflammation. Examples of eye disorders include, but are not limited to glaucoma, macular degeneration, diabetic retinopathy, hereditary retinal degeneration, age-related macular degeneration (e.g., non-exudative age-related macular degeneration, exudative (wet) AMD), macular edema, ocular rosacea, amblyopia, cataracts, dry eye, iritis, photoreceptor degeneration, retinitis pigmentosa, retrobulbar optic neuritis, loss of conjunctival cells, loss of lacrimal gland cells, central or branch retinal artery occlusions, ocular hypertension, neurodegenerative disorders of the retina and optic nerve head, and uveitis. Neurodegenerative disorders of the retina and optic nerve head, include, but are not limited to atrophic macular degeneration; retinitis pigmentosa; iatrogenic retinopathy; retinal tears and holes; diabetic retinopathy; sickle cell retinopathy; retinal vein and artery occlusion; and optic neuropathy.

The term “subject” includes living organisms capable of suffering from or at risk of an eye disorder (e.g., mammals). Examples of subjects at risk may also include diabetics and those with a genetic predisposition to an eye disorder. Examples of subjects include humans, dogs, cats, horses, cows, goats, rats and mice. The term “subject” also includes include transgenic species. In one embodiment, the subject may be a human.

The term “energy in the eye” includes any form of energy in the eye. Examples of energy in the eye include the energy generated by kinases though phosphorylation, e.g., the creatine kinase/creatine phosphate energy system.

The term “modulation” includes the up or down regulation of pathways or systems such as, but not limited to, the creatine kinase/creatine phosphate energy system. Examples of modulation include, but are not limited to, for example, increases in the level of phosphorylation by creatine kinase. For example, the level of phosphorylation may be increased by at least about 5% or greater, by at least about 10% or greater, by at least about 15% or greater, by at least about 20% or greater, by at least about 30% or greater, by at least about 40% or greater, by at least about 50% or greater or by at least about 75% or greater.

The term “administration” includes routes of administration which allow the creatine compounds to perform their intended function(s) of preventing, ameliorating, arresting, and/or eliminating eye disorders in a subject. Examples of routes of administration which may be used include injection, topical, oral, subcutaneous, intraocular, intravenous, parenterally, intraperitoneally, inhalation and transdermal routes of administration. Depending on the route of administration, the compound may be coated with or in a material to protect it from the natural conditions which may detrimentally effect its ability to perform its intended function. The administration of the compound may be done at dosages and for periods of time effective to reduce, ameliorate or eliminate the symptoms of the eye disorder being treated. Dosage regimes may be adjusted for purposes of improving the therapeutic or prophylactic response of the compound. For example, several divided doses may be administered daily or the dose may be proportionally reduced as indicated by the exigencies of the therapeutic situation. The compounds of the invention may be administered in a variety of ways, including all forms of local delivery to the eye, such as subconjunctival injections or implants, intravitreal injections or implants, sub-Tenon's injections or implants, incorporation in surgical irrigating solutions, etc. Suitable pharmaceutical vehicles or dosage forms for injectable compositions, implants, and systemic administration are known. The compounds may be administered topically to the eye and can be formulated into a variety of topically administrable ophthalmic compositions, such as solutions, suspensions, gels or ointments.

The term “therapeutically effective amount” or “effective amount” includes an amount of the compound that may be sufficient in treating or preventing an eye disorder. A therapeutically effective amount may be determined on an individual basis and will be based, in part, on the severity of the symptoms and the activity of the specific creatine compound. Thus, a therapeutically effective amount of a creatine compound may be determined by one of ordinary skill in the art using no more than routine experimentation in clinical management.

In another embodiment, the creatine compounds of the invention may be administered in combination with one or more additional methods of treating eye disorders.

The term “in combination with one or more additional methods of treating eye disorders” include simultaneous administration of (or treatment with) the additional method(s) of treating eye disorders with the creatine compound; administration of (or treatment with) the creatine compound first; followed by the additional method(s) of treating eye disorders; and administration of (or treatment with) the additional method(s) of treating eye disorders first, followed by the creatine compound second. Any of the therapeutically useful method known in the art for treating a particular eye disorder can be used in the methods of the invention.

Examples of additional methods of treating eye disorders include, but are not limited to the methods described.

For example, methods for treatment of non-exudative age-related macular degeneration (dry AMD) include the administration of luten and sub-acute diode laser treatment.

Methods of treatment of exudative (wet) AMD includes laser photocoagulation and photodynamic therapy.

Methods of treating retinopathies, such as, for example diabetic retinopathy, include oral hypoglycemics and laser treatments (e.g., focal and pan-retinal laser photocoagulation).

Examples of treatments for hereditary retinal degeneration, such as retinitis pigmentosa (e.g., both hereditary and sporadic cases), Usher's syndrome, Fundus Albipunctatus, and Stargardt's Disease include administering Vitamin A supplements, and potentially, gene therapies in the future.

Methods of treatment of field loss (e.g., field loss due to glaucoma), include, but are not limited to trabeculoplasty, iridectomy, iridotomy, filtration surgery, administration of drugs that increase aqueous outflow through the trabecular meshwork or through the uveal tract, and administration of drugs that decrease aqueous production.

Examples of methods of treatment for retrobulbar optic neuritis include the administration of steroids.

Methods for treating central or branch retinal artery occlusions include the administration of anticoagulants and clot busting drugs as well as laser treatments. Central or branch vein occlusions may be treated using similar methods.

Photoreceptor degeneration, such as that associated with chronic macular edema, is generally treated by the administration of steroids. For the treatment of toxic retinopathies due to systemic drugs, a method of treatment includes withdrawal of the drug.

Examples of methods of treating photoreceptor degeneration associated with rhegmatogenous retinal detachment, include repairing the detachment.

Methods for treating photoreceptor degeneration associated with non-rhegmatogenous retinal detachment, include eliminating the cause of the exudative detachment (e.g., by a subretinal neurovascular net).

Methods of treating a loss of conjunctival cells or a loss of lacrimal gland cells in severe allergic reactions (e.g., Stevens Johnson syndrome) include withdrawing the drug causing the allergic reaction or by administering steroids.

Methods of treating a loss of visual field owing to ischemia, tumor pressure, or radiation-induced damage of the visual cortex of the occipital lobe, the optic radiation, the lateral geniculate, the optic tracts, chiasm, or the optic nerve, include the administration of steroids or clot busting drugs, and, when appropriate, removing tumors.

### II. Creatine Compounds

Creatine compounds useful in the present invention include compounds which modulate one or more of the structural or functional components of the creatine kinase/phosphocreatine system. Compounds which are effective for this purpose include creatine, creatine phosphate and analogs thereof, compounds which mimic their activity, and salts of these compounds. Exemplary creatine compounds are described below.

The term “creatine compounds” includes compounds of the general formula I:

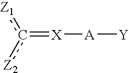

and pharmaceutically acceptable salts thereof, where:
a) Y is selected from the group consisting of: —CO₂H, —NHOH, —NO₂, —SO₃H, —C(═O)NHSO₂J and —P(═O)(OH)(OJ), wherein J is selected from the group consisting of: hydrogen, C₁-C₆ straight chain alkyl, C₃-C₆ branched alkyl, C₂-C₆ alkenyl, C₃-C₆ branched alkenyl, and aryl;
b) A is selected from the group consisting of: C, CH, C₁-C₅ alkyl, C₂-C₅ alkenyl, C₂-C₅ alkynyl, and C₁-C₅ alkoyl chain, each having 0-2 substituents which are selected independently from the group consisting of:
   1) K, where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;
   2) an aryl group selected from the group consisting of: a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy; and
   3) —NH-M, wherein M is selected from the group consisting of: hydrogen, C₁-C₄ alkyl, C₂-C₄ alkenyl, C₁-C₄ alkoyl, C₃-C₄ branched alkyl, C₃-C₄ branched alkenyl, and C₄ branched alkoyl;
c) X is selected from the group consisting of NR₁, CHR₁, CR₁, O and S, wherein R₁ is selected from the group consisting of:
   1) hydrogen;
   2) K¹ where K¹ is selected from the group consisting of: C₁-C₆ straight alkyl, C₂-C₆ straight alkenyl, C₂-C₆ straight alkoyl, C₃-C₆ branched alkyl, C₃-C₆ branched alkenyl, and C₄-C₆ branched alkoyl, K¹ having 0-2 substituents independently selected from the group consisting of: bromo, chloro, epoxy and acetoxy;
   3) an aryl group selected from the group consisting of a 1-2 ring carbocycle and a 1-2 ring heterocycle, wherein the aryl group contains 0-2 substituents independently selected from the group consisting of: —CH₂L¹ and —COCH₂L¹ where L¹ is independently selected from the group consisting of: bromo, chloro, epoxy and acetoxy;
   4) a C₅-C₉ α-amino-ω-methyl-ω-adenosylcarboxylic acid attached via the ω-methyl carbon;
   5) a C₅-C₉ α-amino-ω-aza-ω-methyl-w-adenosylcarboxylic acid attached via the ω-methyl carbon; and
   6) a C₅-C₉ α-amino-ω-thia-ω-methyl-w-adenosylcarboxylic acid attached via the w-methyl carbon;
d) Z₁ and Z₂ are chosen independently from the group consisting of: ═O, —NHR₂, —CH₂R₂, —NR₂OH; wherein Z₁ and Z₂ may not both be ═O and wherein R₂ is selected from the group consisting of:
   1) hydrogen;
   2) K², where K² is selected from the group consisting of: C₁-C₆ straight alkyl; C₂-C₆ straight alkenyl, C₂-C₆ straight alkoyl, C₃-C₆ branched alkyl, C₃-C₆ branched alkenyl, and C₄-C₆ branched alkoyl, K² having 0-2 substituents independently selected from the group consisting of: bromo, chloro, epoxy and acetoxy;
   3) an aryl group selected from the group consisting of a 1-2 ring carbocycle and a 1-2 ring heterocycle, wherein the aryl group contains 0-2 substituents independently selected from the group consisting of: —CH₂L² and —COCH₂L² where L² is independently selected from the group consisting of: bromo, chloro, epoxy and acetoxy;
   4) a C₄-C₈ α-amino-carboxylic acid attached via the ω-carbon;
   5) B, wherein B is selected from the group consisting of: —CO₂H, —NHOH, —SO₃H, —NO₂, OP(═O)(OH)(OJ¹) and —P(═O)(OH)(OJ¹), wherein J¹ is selected from the group consisting of: hydrogen, C₁-C₆ straight alkyl, C₃-C₆ branched alkyl, C₂-C₆ alkenyl, C₃-C₆ branched alkenyl, and aryl, wherein B is optionally connected to the nitrogen via a linker selected from the group consisting of: C₁-C₂ alkyl, C₂ alkenyl, and C₁-C₂ alkoyl;
   6) -D-E, wherein D is selected from the group consisting of: C₁-C₃ straight alkyl, C₃ branched alkyl, C₂-C₃ straight alkenyl, C₃ branched alkenyl, C₁-C₃ straight alkoyl, aryl and aroyl; and E is selected from the group consisting of: —(PO₃)ₙNMP, where n is 0-2 and NMP is ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q¹, where m is 0-3 and Q¹ is a ribonucleoside connected via the ribose or the aromatic ring of the base; and an aryl group containing 0-3 substituents chosen independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₂-C₆ straight alkoyl, C₃-C₆ branched alkyl, C₃-C₆ branched alkenyl, C₄-C₆ branched alkoyl, wherein E may be attached to any point to D, and if D is alkyl or alkenyl, D may be connected at either or both ends by an amide linkage; and
   7) -E¹, wherein E¹ is selected from the group consisting of —(PO₃)ₙNMP, where n is 0-2 and NMP is a ribonucleotide monophosphate connected via the 5′-phosphate, 3′-phosphate or the aromatic ring of the base; —[P(═O)(OCH₃)(O)]ₘ-Q², where m is 0-3 and Q² is a ribonucleoside connected via the ribose or the aromatic ring of the base; —[P(═O)(OH)(CH₂)]ₘ-Q³, where m is 0-3 and Q³ is a ribonucleoside connected via the ribose or the aromatic ring of the base; and an aryl group containing 0-3 substituents chose independently from the group consisting of: Cl, Br, epoxy, acetoxy, —OG¹, —C(═O)G¹, and —CO₂G¹, where G¹ is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl; and if E¹ is aryl, E¹ may be connected by an amide linkage;
e) if R¹ and at least one R₂ group are present, R¹ may be connected by a single or double bond to an R₂ group to form a cycle of 5 to 7 members;
f) if two R₂ groups are present, they may be connected by a single or a double bond to form a cycle of 4 to 7 members; and
g) if R₁ is present and Z₁ or Z₂ is selected from the group consisting of —NHR₂, —CH₂R₂ and —NR₂OH, then R₁ may be connected by a single or double bond to the carbon or nitrogen of either Z₁ or Z₂ to form a cycle of 4 to 7 members.

Creatine (also known as N-(aminoiminomethyl)-N-methylglycine; methylglycosamine or N-methyl-guanido acetic acid) is a well-known substance. (See, The Merck Index, Eleventh Edition, No. 2570 (1989).

Creatine is phosphorylated chemically or enzymatically by creatine kinase to generate creatine phosphate, which also is well-known (see, The Merck Index, No. 7315). Both creatine and creatine phosphate (phosphocreatine) can be extracted from animal tissue or synthesized chemically. Both are commercially available.

Cyclocreatine is an essentially planar cyclic analog of creatine. Although cyclocreatine is structurally similar to creatine, the two compounds are distinguishable both kinetically and thermodynamically. Cyclocreatine is phosphorylated efficiently by creatine kinase in the forward reaction both in vitro and in vivo. Rowley, G. L., J. Am. Chem. Soc. 93: 5542-5551 (1971); McLaughlin, A. C. et. al., J. Biol. Chem. 247, 4382-4388 (1972).

The phosphorylated compound phosphocyclocreatine is structurally similar to phosphocreatine; however, the phosphorous-nitrogen (P-N) bond of cyclocreatine phosphate is more stable than that of phosphocreatine. LoPresti, P. and M. Cohn, Biochem. Biophys. Acta 998: 317-320 (1989); Annesley, T. M. and J. B. Walker, J. Biol. Chem. 253; 8120-8125, (1978); Annesley, T. M. and J. B. Walker, Biochem. Biophys. Res. Commun. 74:185-190 (1977).

3-Guanidinopropionic acid (3-GPA) is an endogenous metabolite found in animals and humans (Hiraga et. al., J. of Chromatography vol 342, 269-275, 1985; Watanabe et. al., Guanidines edited by Mori et. al., Plenum, N.Y., 49-58, 1983). The compound is available from Sigma chemicals and is an extensively studied analog of creatine.

Guanidino acetate is yet another analog of creatine and is a precursor of creatine in its biosynthetic pathway.

Guanidino benzoic acids are structurally related to creatine. Also compounds that attach amino acid like molecules covalently to creatine are creatine compounds of interest. Examples are creatine-ascorbate and creatine-pyruvate. Other types of molecules could be covalently attached.

Creatine analogs and other agents which act to interfere with the activity of creatine biosynthetic enzymes or with the creatine transporter are useful in the present method of treating or preventing eye disorders. Thus the effects of such compounds can be direct or indirect, operating by mechanisms including, but not limited to, influencing the uptake or biosynthesis of creatine, the function of the creatine phosphate shuttle, enzyme activity, or the activity of associated enzymes, or altering the levels of substrates or products of a reaction to alter the velocity of the reaction.

Creatine, creatine phosphate and many creatine analogs are commercially available. Additionally, analogs of creatine may be synthesized using conventional techniques. For example, creatine can be used as the starting material for synthesizing at least some of the analogs encompassed by formula I. Appropriate synthesis reagents, e.g. alkylating, alkenylating or alkynylating agents may be used to attach the respective groups to target sites. Alternatively, reagents capable of inserting spacer groups may be used to alter the creatine structure. Sites other than the target site are protected using conventional protecting groups while the desired sites are being targeted by synthetic reagents.

If the creatine analog contains a ring structure, then the analog may be synthesized in a manner analogous to that described for cyclocreatine (Wang, T., J. Org. Chem. 39:3591-3594 (1974)). The various other substituent groups may be introduced before or after the ring is formed.

Many creatine analogs have been previously synthesized and described (Rowley et al., J. Am. Chem. Soc. 93:5542-5551 (1971); McLaughlin et al., J. Biol. Chem. 247:4382-4388 (1972); Nguyen, A. C. K., “Synthesis and enzyme studies using creatine analogs”, Thesis, Dept. of Pharmaceutical Chemistry, Univ. Calif., San Francisco (1983); Lowe et al., J. Biol. Chem. 225:3944-3951 (1980); Roberts et al., J. Biol. Chem. 260:13502-13508 (1985); Roberts et al. Arch. Biochem. Biophys. 220:563-571 (1983), and Griffiths et al., J. Biol. Chem. 251:2049-2054 (1976)). The contents of all of the aforementioned references are expressly incorporated by reference. Further to the aforementioned references, Kaddurah-Daouk et al. (WO92/08456; WO90/09192; U.S. Pat. No. 5,324,731; U.S. Pat. No. 5,321,030) also provide citations for the synthesis of a plurality of creatine analogs. Also the synthesis of creatine-pyruvate and creatine ascorbate has been described in a series of patents WPI 98-481123/199841; WPI 98-457997/199840; WPI 98-387651/199833). The contents of all the aforementioned references and patents are incorporated herein by reference.

Creatine compounds which currently are available or have been synthesized include, for example, creatine, β-guanidinopropionic acid, creatine monohydrate, guanidinoacetic acid, creatine phosphate disodium salt, cyclocreatine, homocyclocreatine, phosphinic creatine, homocreatine, ethylcreatine, cyclocreatine phosphate dilithium salt, guanidinoacetic acid phosphate disodium salt, 4 guanidino benzoic acid and derivatives, creatine pyruvate, creatine ascorbate, creatine citrate among others.

Creatine phosphate compounds also can be synthesized chemically or enzymatically. The chemical synthesis is well known. Annesley, T. M. Walker, J. B., Biochem. Biophys. Res. Commun., (1977), 74, 185-190; Cramer, F., Scheiffele, E., Vollmar, A., Chem. Ber., (1962), 95, 1670-1682.

Salts of the products may be exchanged to other salts using standard protocols. The enzymatic synthesis utilizes the creatine kinase enzyme, which is commercially available, to phosphorylate the creatine compounds. ATP is required by creatine kinase for phosphorylation, hence it needs to be continuously replenished to drive the reaction forward. It is necessary to couple the creatine kinase reaction to another reaction that generates ATP to drive it forward. The purity of the resulting compounds can be confirmed using known analytical techniques including ¹H NMR, ¹³CNMR Spectra, Thin layer chromatography, HPLC and elemental analysis.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, the term alkyl includes both “unsubstituted alkyls” and “substituted alkyls,” the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids.

The term “alkoyl” denotes an alkyl group as defined above, connected through a carbonyl group to the parent molecular residue. Examples include, but are not limited to formyl, acetyl, propionyl, butyryl, iso-butyryl, pivaloyl, and the like.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles,” “heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond.

For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls,” the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls,” the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” includes an alkyl group, as defined above, but having from one to five carbon atoms in its backbone structure. “Lower alkenyl” and “lower alkynyl” have chain lengths of, for example, 2-5 carbon atoms.

The term “acyl” includes compounds and moieties which contain the acyl radical (CH₃CO—) or a carbonyl group. It includes substituted acyl moieties. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthyl carboxy, etc.

The terms “alkoxyalkyl,” “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term includes “alkyl amino” which comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

The term “carbonyl” or “carboxy” includes compounds and moieties which contain a carbon connected with a double bond to an oxygen atom. The carbonyl can be further substituted with any moiety which allows the compounds of the invention to perform its intended function. For example, carbonyl moieties may be substituted with alkyls, alkenyls, alkynyls, aryls, alkoxy, aminos, etc. Examples of moieties which contain a carbonyl include aldehydes, ketones, carboxylic acids, amides, esters, anhydrides, etc.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “hydroxy” or “hydroxyl” includes groups with an —OH or —O^{—}.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” refer to two or more cyclic rings (e.g., cycloalkyls, cycloalkenyls, cycloalkynyls, aryls and/or heterocyclyls) in which two or more carbons are common to two adjoining rings, e.g., the rings are “fused rings.” Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, arylalkylaminocarbonyl, alkenylaminocarbonyl, alkylcarbonyl, arylcarbonyl, arylalkyl carbonyl, alkenylcarbonyl, aminocarbonyl, alkylthiocarbonyl, alkoxyl, phosphate, phosphonato, phosphinato, cyano, amido, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

### III. Pharmaceutical Compositions

The present invention also pertains, at least in part, to pharmaceutical compositions comprising an effective amount of a creatine compound and a pharmaceutically acceptable carrier. The compositions may be administered orally, by injection, intraocularly, topically (e.g., as an emulsion, gel, solution, suspension, tablet, or ointment), or by any other route which leads to the eye disorder being treated. The composition may also be suitable for opthalmic administration.

The phrase “pharmaceutically acceptable carrier” includes a pharmaceutically acceptable material, composition or vehicle, such as a liquid or solid filler, diluent, excipient, solvent or encapsulating material, involved in carrying or transporting the creatine compound within or to the subject such that it can performs its intended function, e.g. to treat an eye disorder. Each carrier must be “acceptable” in the sense of being compatible with the other ingredients of the formulation and not injurious to the subject. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds of the invention.

Formulations of the invention include those suitable for oral, nasal, opthalmic, topical, transdermal, buccal, sublingual and/or parenteral administration. The formulations may conveniently be presented in unit dosage form and may be prepared by any methods well known in the art of pharmacy. Methods of preparing these formulations or compositions include the step of bringing into association a creatine compound with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Formulations of the invention suitable for oral administration may be in the form of capsules, cachets, pills, wafers, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound of the invention may also be administered as a bolus, electuary or paste.

In solid dosage forms of the invention for oral administration (capsules, tablets, wafers, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral or opthalmic administration of a creatine compound include opthalmically and pharmaceutically acceptable emulsions, microemulsions, solutions, suspensions, syrups and elixirs. In addition to the active ingredient, the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert dilutents, oral or opthalmic compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for transdermal administration of compounds of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to creatine monohydrate and/or an anti-inflammatory compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a creatine compound to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

The creatine compound of the present invention may be administered in combination with a pharmaceutically acceptable carrier. The pharmaceutical carrier may also be suitable for administration to the eye.

Pharmaceutical compositions of this invention suitable for parenteral administration comprise a creatine compound in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound, it is desirable to slow the absorption of the compound from subcutaneous or intramuscular injection. This may be accomplished by the use of a liquid suspension of crystalline or amorphous material having poor water solubility. The rate of absorption of the compound then depends upon its rate of dissolution which, in turn, may depend upon crystal size and crystalline form. Alternatively, delayed absorption of a parenterally-administered compound form is accomplished by dissolving or suspending the compound in an oil vehicle.

Injectable depot forms are made by forming microencapsule matrices of the compounds of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the invention may be given orally, parenterally or topically. They are, of course, given by forms suitable for each administration route. For example, they are administered in tablets or capsule form, by injection, inhalation, eye drops, eye lotion, gel, ointment, suppository, etc. administration by injection, infusion or inhalation; topical by lotion or ointment; and rectal by suppositories. Oral or opthalmic administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

Compositions for opthalnic administration may be formulated as solutions for both topical administration or for injection. For example, solutions of the formulation may be applied in drop form to the eye. The compositions may also be formulated as gels, suspensions or ointments for application to the eye.

In a further embodiment, the present invention pertains, at least in part, to a pharmaceutical composition to treat an eye disorder which is suitable for opthalmic administration comprising an effective amount of a creatine compound and an opthalmically acceptable carrier. In one embodiment, the composition is suitable for administration topically or by injection. In another embodiment, the composition is a gel, emulsion, solution, suspension or ointment.

The phrase “opthalmically acceptable carrier” includes carriers that are compatible with administration to the eye whether topically or by injection to the eye. Typical formulations for opthalmic administration may be aqueous solutions and contain buffers (e.g. borate) to maintain an appropriate pH, salts, water soluble polymers, thickening agents, preservatives and other additives that make the formulation appropriate for opthalmic administration.

### Equivalents

Those skilled in the art will recognize, or be able to ascertain using no more than routine experimentation, many equivalents to the specific embodiments of the invention described herein. Such equivalents are intended to be encompassed by the following claims.

The entire contents of all references, patents, and patent applications cited herein are expressly incorporated by reference.

1. A method for treating an eye disorder in a subject, comprising administering to said subject an effective amount of a creatine compound such that said eye disorder is treated.

2. The method of claim 1, wherein said eye disorder is selected from the group consisting of glaucoma, macular degeneration, diabetic retinopathy, age-related macular degeneration, macular edema, ocular rosacea, amblyopia, cataracts, dry eye, iritis, retinitis pigmentosa and uveitis.

3. (canceled)

4. (canceled)

5. The method of claim 1, wherein said subject is a mammal.

6. The method of claim 5, wherein said subject is a human.

7. The method of claim claim 1, wherein said creatine compound is of the formula: 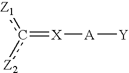
and pharmaceutically acceptable salts thereof, wherein:
a) Y is selected from the group consisting of: —CO₂H, —NHOH, —NO₂, —SO₃H, —C(═O)NHSO₂J and —P(═O)(OH)(OJ), wherein J is selected from the group consisting of: hydrogen, C₁-C₆ straight chain alkyl, C₃-C₆ branched alkyl, C₂-C₆ alkenyl, C₃-C₆ branched alkenyl, and aryl;
b) A is selected from the group consisting of: C, CH, C₁-C₅ alkyl, C₂-C₅ alkenyl, C₂-C₅ alkynyl, and C₁-C₅ alkoyl chain, each having 0-2 substituents which are selected independently from the group consisting of:
1) K, where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;
2) an aryl group selected from the group consisting of: a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy; and
3) —NH-M, wherein M is selected from the group consisting of: hydrogen, C₁-C₄ alkyl, C₂-C₄ alkenyl, C₁-C₄ alkoyl, C₃-C₄ branched alkyl, C₃-C₄ branched alkenyl, and C₄ branched alkoyl;
c) X is selected from the group consisting of NR₁, CHR₁, CR₁, O and S, wherein R₁ is selected from the group consisting of:
1) hydrogen;
2) K¹ where K¹ is selected from the group consisting of: C₁-C₆ straight alkyl, C₂-C₆ straight alkenyl, C₂-C₆ straight alkoyl, C₃-C₆ branched alkyl, C₃-C₆ branched alkenyl, and C₄-C₆ branched alkoyl, K¹ having 0-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L¹ and —COCH₂L¹ where L¹ is independently selected from the group consisting of: bromo, chloro, epoxy and acetoxy;
4) a C₅-C₉ α-amino-ω-methyl-ω-adenosylcarboxylic acid attached via the co-methyl carbon;
5) a C₅-C₉ α-amino-ω-aza-ω-methyl-w-adenosylcarboxylic acid attached via the ω-methyl carbon; and
6) a C₅-C₉ α-amino-ω-thia-ω-methyl-w-adenosylcarboxylic acid attached via the w-methyl carbon;
d) Z₁ and Z₂ are chosen independently from the group consisting of: ═O, —NHR₂, —CH₂R₂, —NR₂OH; wherein Z₁ and Z₂ may not both be ═O and wherein R₂ is selected from the group consisting of:
1) hydrogen;
2) K , where K² is selected from the group consisting of: C₁-C₆ straight alkyl; C₂-C₆ straight alkenyl, C₂-C₆ straight alkoyl, C₃-C₆ branched alkyl, C₃-C₆ branched alkenyl, and C₄-C₆ branched alkoyl, K² having 0-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L and —COCH₂L² where L² is independently selected from the group consisting of: bromo, chloro, epoxy and acetoxy;
4) a C₄-C₈ α-amino-carboxylic acid attached via the ω-carbon;
5) B, wherein B is selected from the group consisting of: —CO₂H, —NHOH, —SO₃H, —NO₂, OP(═O)(OH)(OJ¹) and —P(═O)(OH)(OJ¹), wherein J¹ is selected from the group consisting of: hydrogen, C₁-C₆ straight alkyl, C₃-C₆ branched alkyl, C₂-C₆ alkenyl, C₃-C₆ branched alkenyl, and aryl, wherein B is optionally connected to the nitrogen via a linker selected from the group consisting of: C₁-C₂ alkyl, C₂ alkenyl, and C₁-C₂ alkoyl;
6) -D-E, wherein D is selected from the group consisting of: C₁-C₃ straight alkyl, C₃ branched alkyl, C₂-C₃ straight alkenyl, C₃ branched alkenyl, C₁-C₃ straight alkoyl, aryl and aroyl; and E is selected from the group consisting of: —(PO₃)ₙNMP, where n is 0-2 and NMP is ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q¹, where m is 0-3 and Q¹ is a ribonucleoside connected via the ribose or the aromatic ring of the base; and an aryl group containing 0-3 substituents chosen independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₂-C₆ straight alkoyl, C₃-C₆ branched alkyl, C₃-C₆ branched alkenyl, C₄-C₆ branched alkoyl, wherein E may be attached to any point to D, and if D is alkyl or alkenyl, D may be connected at either or both ends by an amide linkage; and
7) -E¹, wherein E¹ is selected from the group consisting of —(PO₃)ₙNMP, where n is 0-2 and NMP is a ribonucleotide monophosphate connected via the 5′-phosphate, 3′-phosphate or the aromatic ring of the base; —[P(═O)(OCH₃)(O)]ₘ-Q², where m is 0-3 and Q² is a ribonucleoside connected via the ribose or the aromatic ring of the base; —[P(═O)(OH)(CH₂)]ₘ-Q³, where m is 0-3 and Q³ is a ribonucleoside connected via the ribose or the aromatic ring of the base; and an aryl group containing 0-3 substituents chose independently from the group consisting of: Cl, Br, epoxy, acetoxy, —OG¹, —C(═O)G¹, and —CO₂G¹, where G¹ is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl; and if E¹ is aryl, E¹ maybe connected by an amide linkage;
e) if R₁ and at least one R₂ group are present, R₁ may be connected by a single or double bond to an R₂ group to form a cycle of 5 to 7 members;
f) if two R₂ groups are present, they may be connected by a single or a double bond to form a cycle of 4 to 7 members; and
g) if R₁ is present and Z₁ or Z₂ is selected from the group consisting of —NHR₂, —CH₂R₂ and —NR₂OH, then R₁ may be connected by a single or double bond to the carbon or nitrogen of either Z₁ or Z₂ to form a cycle of 4 to 7 members.

8. The method of claim 1, wherein said creatine compound is creatine, creatine phosphate, cyclocreatine, cyclocreatine phosphate, creatine pyruvate, creatine ascorbate, creatine monohydrate, homocyclocreatine, creatine citrate or 3-guanidinopropionic acid.

9.-17. (canceled)

18. The method of claim 1, further comprising administering said compound in combination with a pharmaceutically acceptable carrier.

19. The method of claim 18, wherein the said pharmaceutically acceptable carrier is suitable for administration to the eye.

20. A method for modulating energy in an eye of a subject, comprising administering to said subject an effective amount of a creatine compound, such that energy is modulated in the eye.

21. (canceled)

22. (canceled)

23. The method of claim 20, wherein said subject is a mammal.

24. The method of claim 23, wherein said subject is a human.

25. The method claim 20, wherein said subject is suffering from an eye disorder.

26. The method of claim 25, wherein said eye disorder is selected from the group consisting of glaucoma, macular degeneration, diabetic retinopathy, age-related macular degeneration, macular edema, ocular rosacea, amblyopia, cataracts, dry eye, iritis, retinitis pigmentosa and uveitis.

27. The method of claim of claim 20, wherein said creatine compound is of the formula: 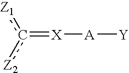
and pharmaceutically acceptable salts thereof, wherein:
a) Y is selected from the group consisting of: —CO₂H, —NHOH, —NO₂, —SO₃H, —C(═O)NHSO₂J and —P(═O)(OH)(OJ), wherein J is selected from the group consisting of: hydrogen, C₁-C₆ straight chain alkyl, C₃-C₆ branched alkyl, C₂-C₆ alkenyl, C₃-C₆ branched alkenyl, and aryl;
b) A is selected from the group consisting of: C, CH, C₁-C₅ alkyl, C₂-C₅ alkenyl, C₂-C₅ alkynyl, and C₁-C₅ alkoyl chain, each having 0-2 substituents which are selected independently from the group consisting of:
1) K, where K is selected from the group consisting of: C₁-C₆ straight alkyl, C₂-C₆ straight alkenyl, C₁-C₆ straight alkoyl, C₃-C₆ branched alkyl, C₃-C₆ branched. alkenyl, and C₄-C₆ branched alkoyl, K having 0-2 substituents independently selected from the group consisting of: bromo, chloro, epoxy and acetoxy;
2) an aryl group selected from the group consisting of: a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy; and
3) —NH-M, wherein M is selected from the group consisting of: hydrogen, C₁-C₄ alkyl, C₂-C₄ alkenyl, C₁-C₄ alkoyl, C₃-C₄ branched alkyl, C₃-C₄ branched alkenyl, and C₄ branched alkoyl;
c) X is selected from the group consisting of NR₁, CHR₁, CR₁, O and S, wherein R₁ is selected from the group consisting of:
1) hydrogen;
2) K¹ where K¹ is selected from the group consisting of: C₁-C₆ straight alkyl, C₂-C₆ straight alkenyl, C₂-C₆ straight alkoyl, C₃-C₆ branched alkyl, C₃-C₆ branched alkenyl, and C₄-C₆ branched alkoyl, K¹ having 0-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L¹ and —COCH₂L¹ where L¹ is independently selected from the group consisting of: bromo, chloro, epoxy and acetoxy;
4) a C₅-C₉ α-amino-ω-methyl-ω-adenosylcarboxylic acid attached via the ω-methyl carbon;
5) a C₅-C₉ α-amino-ω-aza-ω-methyl-w-adenosylcarboxylic acid attached via the ω-methyl carbon; and
6) a C₅-C₉ α-amino-ω-thia-ω-methyl-w-adenosylcarboxylic acid attached via the w-methyl carbon;
d) Z₁ and Z₂ are chosen independently from the group consisting of: ═O, —NHR₂, —CH₂R₂, —NR₂OH; wherein Z₁ and Z₂ may not both be ═O and wherein R₂ is selected from the group consisting of:
1) hydrogen;
2) K², where K² is selected from the group consisting of: C₁-C₆ straight alkyl; C₂-C₆ straight alkenyl, C₂-C₆ straight alkoyl, C₃-C₆ branched alkyl, C₃-C₆ branched alkenyl, and C₄-C₆ branched alkoyl, K² having 0-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L² and —COCH₂L²where L² is independently selected from the group consisting of: bromo, chloro, epoxy and acetoxy;
4) a C₄-C₈ α-amino-carboxylic acid attached via the ω-carbon;
5) B, wherein B is selected from the group consisting of: —CO₂H, —NHOH, —SO₃H, —NO₂, OP(═O)(OH)(OJ¹) and —P(═O)(OH)(OJ¹), wherein J¹ is selected from the group consisting of: hydrogen, C₁-C₆ straight alkyl, C₃-C₆ branched alkyl, C₂-C₆ alkenyl, C₃-C₆ branched alkenyl, and aryl, wherein B is optionally connected to the nitrogen via a linker selected from the group consisting of: C₁-C₂ alkyl, C₂ alkenyl, and C₁-C₂ alkoyl;
6) -D-E, wherein D is selected from the group consisting of: C₁-C₃ straight alkyl, C₃ branched alkyl, C₂-C₃ straight alkenyl, C₃ branched alkenyl, C₁-C₃ straight alkoyl, aryl and aroyl; and E is selected from the group consisting of: —(PO3)ₙNMP, where n is 0-2 and NMP is ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q¹, where m is 0-3 and Q¹ is a ribonucleoside connected via the ribose or the aromatic ring of the base; and an aryl group containing 0-3 substituents chosen independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₂-C₆ straight alkoyl, C₃-C₆ branched alkyl, C₃-C₆ branched alkenyl, C₄-C₆ branched alkoyl, wherein E may be attached to any point to D, and if D is alkyl or alkenyl, D may be connected at either or both ends by an amide linkage; and
7) -E¹, wherein E¹ is selected from the group consisting of —(PO₃)ₙNMP, where n is 0-2 and NMP is a ribonucleotide monophosphate connected via the 5′-phosphate, 3′-phosphate or the aromatic ring of the base; —[P(═O)(OCH₃)(O)]ₘ-Q², where m is 0-3 and Q² is a ribonucleoside connected via the ribose or the aromatic ring of the base; [P(═O)(OH)(CH₂)]ₘ-Q³, where m is 0-3 and Q³ is a ribonucleoside connected via the ribose or the aromatic ring of the base; and an aryl group containing 0-3 substituents chose independently from the group consisting of: Cl, Br, epoxy, acetoxy, —OG¹, —C(═O)G¹, and —CO₂G¹, where G¹ is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl; and if E¹ is aryl, E¹ may be connected by an amide linkage;
e) if R₁ and at least one R₂ group are present, R₁ may be connected by a single or double bond to an R₂ group to form a cycle of 5 to 7 members;
f) if two R₂ groups are present, they may be connected by a single or a double bond to form a cycle of 4 to 7 members; and
g) if R₁ is present and Z₁ or Z₂ is selected from the group consisting of —NHR₂, —CH₂R₂ and —NR₂OH, then R₁ may be connected by a single or double bond to the carbon or nitrogen of either Z₁ or Z₂ to form a cycle of 4 to 7 members.

28. The method of claim 20, wherein said creatine compound is creatine, creatine phosphate, cyclocreatine, cyclocreatine phosphate, creatine pyruvate, creatine ascorbate, creatine monohydrate, homocyclocreatine, creatine citrate or 3-guanidinopropionic acid.

29.-36. (canceled)

37. The method of claim 20, further comprising administering said compound in combination with a pharmaceutically acceptable carrier.

38. The method of claim 37, wherein the said pharmaceutically acceptable carrier is suitable for administration to the eye.

39. A method of treating glaucoma in a subject, comprising administering to said subject an effective amount of a creatine compound such that said subject is treated.

40.-42. (canceled)

43. The method of claim 39, wherein said subject is a mammal.

44. The method of claim 43, wherein said subject is a human.

45. The method of claim 44, wherein said human has diabetes.

46. The method of claim 39, wherein said creatine compound is of the formula: 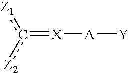
and pharmaceutically acceptable salts thereof, wherein:
a) Y is selected from the group consisting of: —CO₂H, —NHOH, —NO₂, —SO₃H, —C(═O)NHSO₂J and —P(═O)(OH)(OJ), wherein J is selected from the group consisting of: hydrogen, C₁-C₆ straight chain alkyl, C₃-C₆ branched alkyl, C₂-C₆ alkenyl, C₃-C₆ branched alkenyl, and aryl;
b) A is selected from the group consisting of: C, CH, C₁-C₅ alkyl, C₂-C₅ alkenyl, C₂-C₅ alkynyl, and C₁-C₅ alkoyl chain, each having 0-2 substituents which are selected independently from the group consisting of:
1) K, where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;
2) an aryl group selected from the group consisting of: a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy; and
3) —NH-M, wherein M is selected from the group consisting of: hydrogen, C₁-C₄ alkyl, C₂-C₄ alkenyl, C₁-C₄ alkoyl, C₃-C₄ branched alkyl, C₃-C₄ branched alkenyl, and C₄ branched alkoyl;
c) X is selected from the group consisting of NR₁, CHR₁, CR₁, O and S, wherein R₁ is selected from the group consisting of:
1) hydrogen;
2) K¹ where K¹ is selected from the group consisting of: C₁-C₆ straight alkyl, C₂-C₆ straight alkenyl, C₂-C₆ straight alkoyl, C₃-C₆ branched alkyl, C₃-C₆ branched alkenyl, and C₄-C₆ branched alkoyl, K¹ having 0-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L¹ and —COCH₂L¹ where L¹ is independently selected from the group consisting of: bromo, chloro, epoxy and acetoxy;
4) a C₅-C₉ α-amino-ω-methyl-ω-adenosylcarboxylic acid attached via the ω-methyl carbon;
5) a C₅-C₉ α-amino-ω-aza-ω-methyl-w-adenosylcarboxylic acid attached via the ω-methyl carbon; and
6) a C₅-C₉ α-amino-ω-thia-ω-methyl-w-adenosylcarboxylic acid attached via the w-methyl carbon;
d) Z₁ and Z₂ are chosen independently from the group consisting of: ═O, —NHR₂, —CH₂R₂, —NR₂OH; wherein Z₁ and Z₂ may not both be ═O and wherein R₂ is selected from the group consisting of:
1) hydrogen;
2) K², where K² is selected from the group consisting of: C₁-C₆ straight alkyl; C₂-C₆ straight alkenyl, C₂-C₆ straight alkoyl, C₃-C₆ branched alkyl, C₃-C₆ branched alkenyl, and C₄-C₆ branched alkoyl, K² having 0-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L² and —COCH₂L² where L² is independently selected from the group consisting of: bromo, chloro, epoxy and acetoxy;
4) a C₄-C₈ α-amino-carboxylic acid attached via the ω-carbon;
5) B, wherein B is selected from the group consisting of: —CO₂H, —NHOH, —SO₃H, —NO₂, OP(═O)(OH)(OJ¹) and —P(═O)(OH)(OJ¹), wherein J¹ is selected from the group consisting of: hydrogen, C₁-C₆ straight alkyl, C₃-C₆ branched alkyl, C₂-C₆ alkenyl, C₃-C₆ branched alkenyl, and aryl, wherein B is optionally connected to the nitrogen via a linker selected from the group consisting of: C₁-C₂ alkyl, C₂ alkenyl, and C₁-C₂ alkoyl;
6) -D-E, wherein D is selected from the group consisting of: C₁-C₃ straight alkyl, C₃ branched alkyl, C₂-C₃ straight alkenyl, C₃ branched alkenyl, C₁-C₃ straight alkoyl, aryl and aroyl; and E is selected from the group consisting of: —(PO₃)ₙNMP, where n is 0-2 and NMP is ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q¹, where m is 0-3 and Q¹ is a ribonucleoside connected via the ribose or the aromatic ring of the base; and an aryl group containing 0-3 substituents chosen independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₂-C₆ straight alkoyl, C₃-C₆ branched alkyl, C₃-C₆ branched alkenyl, C₄-C₆ branched alkoyl, wherein E may be attached to any point to D, and if D is alkyl or alkenyl, D may be connected at either or both ends by an amide linkage; and
7) -E¹, wherein E¹ is selected from the group consisting of —(PO₃)ₙNMP, where n is 0-2 and NMP is a ribonucleotide monophosphate connected via the 5′-phosphate, 3′-phosphate or the aromatic ring of the base; —[P(═O)(OCH₃)(O)]ₘ-Q², where m is 0-3 and Q² is a ribonucleoside connected via the ribose or the aromatic ring of the base; —[P(═O)(OH)(CH₂)]ₘ-Q³, where m is 0-3 and Q³ is a ribonucleoside connected via the ribose or the aromatic ring of the base; and an aryl group containing 0-3 substituents chose independently from the group consisting of: Cl, Br, epoxy, acetoxy, —OG¹, —C(═O)G¹, and —CO₂G¹, where. G¹ is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl; and if E¹ is aryl, E¹ may be connected by an amide linkage;
e) if R₁ and at least one R₂ group are present, R₁ may be connected by a single or double bond to an R₂ group to form a cycle of 5 to 7 members;
f) if two R₂ groups are present, they may be connected by a single or a double bond to form a cycle of 4 to 7 members; and
g) if R₁ is present and Z₁ or Z₂ is selected from the group consisting of —NHR₂, —CH₂R₂ and —NR₂OH, then R₁ may be connected by a single or double bond to the carbon or nitrogen of either Z₁ or Z₂ to form a cycle of 4 to 7 members.

47. The method of claim 39, wherein said creatine compound is creatine, creatine phosphate, cyclocreatine, cyclocreatine phosphate, creatine pyruvate, creatine ascorbate, creatine monohydrate, homocyclocreatine, creatine citrate or 3-guanidinopropionic acid.

48.-56. (canceled)

57. The method of claim 39, further comprising administering said compound in combination with a pharmaceutically acceptable carrier.

58. The method of claim 57, wherein the said pharmaceutically acceptable carrier is suitable for administration to the eye.

59. A pharmaceutical composition, comprising an effective amount of a creatine compound and a pharmaceutically acceptable carrier, wherein said effective amount is effective for treating an eye disorder.

60. (canceled)

61. (canceled)

62. The pharmaceutical composition claim 59, wherein said composition is suitable for opthalmic administration.

63. A pharmaceutical composition suitable for opthalmic administration, comprising an effective amount of a creatine compound and a opthalmically acceptable carrier, wherein said effective amount is effective for treating an eye disorder.

64. (canceled)

65. (canceled)

66. The pharmaceutical composition of claim 63, wherein said effective amount is an amount effective to treat an eye disorder.

67. The pharmaceutical composition of claim of claim 63, wherein said creatine compound is of the formula: 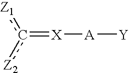
and pharmaceutically acceptable salts thereof, wherein:
a) Y is selected from the group consisting of: —CO₂H, —NHOH, —NO₂, —SO₃H, —C(═O)NHSO₂J and —P(═O)(OH)(OJ), wherein J is selected from the group consisting of: hydrogen, C₁-C₆ straight chain alkyl, C₃-C₆ branched alkyl, C₂-C₆ alkenyl, C₃-C₆ branched alkenyl, and aryl;
b) A is selected from the group consisting of: C, CH, C₁-C₅ alkyl, C₂-C₅ alkenyl, C₂-C₅ alkynyl, and C₁-C₅ alkoyl chain, each having 0-2 substituents which are selected independently from the group consisting of:
1) K, where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;
2) an aryl group selected from the group consisting of: a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy; and
3) —NH-M, wherein M is selected from the group consisting of: hydrogen, C₁-C₄ alkyl, C₂-C₄ alkenyl, C₁-C₄ alkoyl, C₃-C₄ branched alkyl, C₃-C₄ branched alkenyl, and C₄ branched alkoyl;
c) X is selected from the group consisting of NR₁, CHR₁, CR₁, O and S, wherein R₁ is selected from the group consisting of:
1) hydrogen;
2) K¹ where K¹ is selected from the group consisting of: C₁-C₆ straight alkyl, C₂-C₆ straight alkenyl, C₂-C₆ straight alkoyl, C₃-C₆ branched alkyl, C₃-C₆ branched alkenyl, and C₄-C₆ branched alkoyl, K¹ having 0-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L¹ and —COCH₂L¹ where L¹ is independently selected from the group consisting of: bromo, chloro, epoxy and acetoxy;
4) a C₅-C₉ α-amino-ω-methyl-ω-adenosylcarboxylic acid attached via the ω-methyl carbon;
5) a C₅-C₉ α-amino-ω-aza-ω-methyl-w-adenosylcarboxylic acid attached via the ω-methyl carbon; and
6) a C₅-C₉ α-amino-ω-thia-ω-methyl-w-adenosylcarboxylic acid attached via the w-methyl carbon;
d) Z₁ and Z₂ are chosen independently from the group consisting of: ═O, —NHR₂, —CH₂R₂, —NR₂OH; wherein Z₁ and Z₂ may not both be ═O and wherein R₂ is selected from the group consisting of:
1) hydrogen;
2) K², where K² is selected from the group consisting of: C₁-C₆ straight alkyl; C₂-C₆ straight alkenyl, C₂-C₆ straight alkoyl, C₃-C₆ branched alkyl, C₃-C₆ branched alkenyl, and C₄-C₆ branched alkoyl, K² having 0-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L² and —COCH₂L² where L² is independently selected from the group consisting of: bromo, chloro, epoxy and acetoxy;
4) a C₄-C₈ α-amino-carboxylic acid attached via the ω-carbon;
5) B, wherein B is selected from the group consisting of: —CO₂H, —NHOH, —SO₃H, —NO₂, OP(═O)(OH)(OJ¹) and —P(═O)(OH)(OJ¹), wherein J¹ is selected from the group consisting of: hydrogen, C₁-C₆ straight alkyl, C₃-C₆ branched alkyl, C₂-C₆ alkenyl, C₃-C₆ branched alkenyl, and aryl, wherein B is optionally connected to the nitrogen via a linker selected from the group consisting of: C₁-C₂ alkyl, C₂ alkenyl, and C₁-C₂ alkoyl;
6) -D-E, wherein D is selected from the group consisting of: C₁-C₃ straight alkyl, C₃ branched alkyl, C₂-C₃ straight alkenyl, C₃ branched alkenyl, C₁-C₃ straight alkoyl, aryl and aroyl; and E is selected from the group consisting of: —(PO₃)ₙNMP, where n is 0-2 and NMP is ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q¹, where m is 0-3 and Q¹ is a ribonucleoside connected via the ribose or the aromatic ring of the base; and an aryl group containing 0-3 substituents chosen independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₂-C₆ straight alkoyl, C₃-C₆ branched alkyl, C₃-C₆ branched alkenyl, C₄-C₆ branched alkoyl, wherein E may be attached to any point to D, and if D is alkyl or alkenyl, D may be connected at either or both ends by an amide linkage; and
7) -E¹, wherein E¹ is selected from the group consisting of —(PO₃)ₙNMP, where n is 0-2 and NMP is a ribonucleotide monophosphate connected via the 5′-phosphate, 3′-phosphate or the aromatic ring of the base; —[P(═O)(OCH₃)(O)]ₘ-Q2, where m is 0-3 and Q² is a ribonucleoside connected via the ribose or the aromatic ring of the base; —[P(═O)(OH)(CH₂)]ₙ-Q³, where m is 0-3 and Q³ is a ribonucleoside connected via the ribose or the aromatic ring of the base; and an aryl group containing 0-3 substituents chose independently from the group consisting of: Cl, Br, epoxy, acetoxy, —OG¹, —C(═O)G¹, and —CO₂G¹, where G¹ is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl; and if E¹ is aryl, E¹ may be connected by an amide linkage;
e) if R₁ and at least one R₂ group are present, R₁ may be connected by a single or double bond to an R₂ group to form a cycle of 5 to 7 members;
f) if two R₂ groups are present, they may be connected by a single or a double bond to form a cycle of 4 to 7 members; and
g) if R₁ is present and Z₁ or Z₂ is selected from the group consisting of —NHR₂, —CH₂R₂ and —NR₂OH, then R₁ may be connected by a single or double bond to the carbon or nitrogen of either Z₁ or Z₂ to form a cycle of 4 to 7 members.

68. The pharmaceutical composition of claim 63, wherein said creatine compound is selected from the group consisting of creatine, creatine phosphate, cyclocreatine, cyclocreatine phosphate, creatine citrate, creatine pyrivate, creatine ascorbate, creatine monohydrate, homocyclocreatine or 3-guanidinopropionic acid.
